(19) United States
(12) Patent Application Publication
(10) Pub. No.: US 2023/0181348 A1
(43) Pub. Date: Jun. 15, 2023

(54) VACUUM CHAMBER EXTENSION DEVICE FOR PENIS ENLARGEMENT

(71) Applicant: Bruce Gioia, Vancouver (CA)

(72) Inventor: Bruce Gioia, Vancouver (CA)

(21) Appl. No.: 18/106,002

(22) Filed: Feb. 6, 2023

(51) Int. Cl. A61F 5/41

(52) U.S. Cl. CPC A61F 5/41; A61F 2005/412

(57) ABSTRACT

The present disclosure relates to a vacuum chamber extension device for penis enlargement as well as the method of assembling and using the vacuum chamber extension device for penis enlargement. The vacuum chamber extension device includes an elastic polymer sleeve configured to be positioned on the penis shaft, a glans cap configured to be placed over the glans, and a vacuum chamber configured to at least partially enclose the glans cap. The vacuum chamber extension device is safe, easy to use, effective and efficient in use. The vacuum chamber extension device can be easily secured over the penis for the purpose of penis enlargement.

### FIELD

The present invention relates to a penis enlargement device. More particularly, the present disclosure relates to a hollow bell-shaped vacuum chamber with additional components that are used to create an airtight seal for secure attachment over the glans by means of evacuation with an attachment point for applied traction to stretch the penis for the purpose of enlargement.

Penis enlargement methods are generally used by men to increase their penis length size. These methods are also used to correct inherent genetic defects, and environmental influences that cause deviation from what is considered the normal shape of the penis.

A long-standing traditional method for penis enlargement as described by the middle eastern term is to “jelq” Or the act of “jelqing”. Using this method, the semi erect penis is massaged with the hands to squeeze and engorge the anatomy using a stroking action to “milk” more blood into the penis. This effectively stretches the penis along with the expansion of the cellular membranes through the process of hyper engorgement. The limitations to this method are the physical demands of strength and endurance which cause the practitioners' hands to fatigue and tire without the use of leverage to aid in this completely manual technique.

Naturally, this led to the development of simple mechanical means using crude noose like attachments made of rubber, cloth, or leather straps secured on the penis shaft behind the glans for suspension of heavy objects such as rocks or iron weights for applied traction. However, the restrictive nature of these methods disrupts proper blood circulation along with significant discomfort and pain, thereby limiting the duration of exercise to no more than 15 minutes for safety reasons.

Another penis enlargement method or style of attachment is classified as clamp or compression hanging. As the name implies, a clamping device is secured by compressive force onto the shaft of the penis directly behind the glans. This device also includes a strap with a hook for suspending the weight. The limitation of this method is the tight grip required to secure the clamp into position on the penis shaft with subsequent loss of blood circulation, discomfort, and pain. Thus, the standard session duration of one-hour must be broken down into four shorter 15-minute “sets” to safely complete the required time. Otherwise, the risk of oxygen deprivation with subsequent tissue damage and potential cell death become serious matters of concern.

Another penis enlargement method is categorized as vacuum hanging. This method implements the use of a hollow chamber for insertion of the glans which is then held in place by the suction generated by the negative pressure of a vacuum introduced into the chamber.

Use of penis enlargement devices employing/utilizing vacuum force (negative pressure) is well known in the art. For instance, US20160151226A1 as described by Leddy discloses a method and apparatus for penis enlargement. This teaching demonstrates several of the faults common with vacuum chamber devices of this type. The single vacuum port tissue barrier is invariably blocked by the glans as it expands during evacuation. Once the tissue barrier is blocked, no further evacuation of the chamber is possible. The vacuums' negative pressure measured in inches of mercury at the pump will ultimately be limited to an indication of the higher vacuum level created within the air intake opening of the airlock port and therefore is not representative of the vacuum within the chamber itself. Although this disclosure reveals that the horseshoe shaped tissue barrier has air baffles to prevent such blockage, these baffles will also be sealed off by the glans as it expands under a vacuum. Furthermore, without sufficient vacuum within the chamber. The glans will separate from the chamber wall under heavy traction to cavitate and form an air pocket. With greater traction, the air pocket further expands with an increase in negative pressure to potentially pull the skin away from the deeper sub-dermis layer to form a blister. At the same time, the thin “gel flex air seal” Attached at the proximal end of the chamber's tubular lip that is used for sealing against the penis shaft is not sufficient to prevent the sensitive skin of the frenulum from being sucked up into the chamber, and with the application of greater traction, the frenulum swells up with interstitial fluid to form an unsightly puffy mass which is revealed only after removal of the assembly.

U.S. Pat. No. 5,707,341A as described by Mathewuse discloses a penis gripping device. This device demonstrates similar faults as described by Leddy in the previous reference. That being, a single vacuum port or valve aperture, within the chamber body. However, in this case, the glans does not completely fill the chamber and subsequently, a large volume of air remains after evacuation for an unspecified “mild” or “partial” Vacuum. Under applied traction, the air pocket further expands with a corresponding increase in negative pressure to higher and more dangerous levels. To counter this effect, the described art requires a padded containment, with an air bladder, to surround the glans, thereby preventing the glans from “swelling outward”. However, this presents a new problem, that being, reduced contact between the glans and the chamber wall, thereby preventing the necessary friction required to keep the glans from slipping out of the chamber under applied traction. The described means of attaching the padded containment, with air bladder, does not fully surround the glans leaving the delicate urinary meatus exposed to substantially higher vacuum levels with subsequent increased potential of blister formation. Additionally, the thin rubber sheath, that is secured on the chamber body, by stretching over the annular rim of the proximal opening, allows the frenulum to be sucked up into the chamber body thereby causing interstitial fluid to collect and swell into a puffy mass as a result of applied traction when suspending weight from, by means of attachment, as seen in FIG. 3

DE102007017222A1 as described by Jochum discloses a penis extension assembly with frenulum suction cup coupled to lever-action. This device again demonstrates similar faults as described in the prior art by Leddy and Mathewuse. That being, a cup-shaped part 5 with a single opening 12 to the pipe socket 8. In this case, a foam insert 11 or a separate pressure cuff 20 is used to contain the glans and prevent over expansion of the tissues under vacuum. However, the foam insert 11 in no way prevents the single opening 12 from being blocked and thereby limits the vacuum reading to the negative pressure generated within the pipe socket 8 rather than the chamber itself as criticized in the previously referenced examples of prior art. The pressure cuff 20 is designed to include a urethral outlet opening 21 which leaves the most sensitive part of the anatomy, the urinary meatus, exposed to dangerously high vacuum levels with the potential for blister formation under applied traction. Furthermore, the foam insert 11, does not extend the full length of the cup-shaped part 5 which allows the frenulum to be drawn up into the wider proximal opening of the suction cup thereby causing interstitial fluid to collect and swell the frenulum up into a puffy mass. The pressure cuff 20 provides no protection whatsoever to prevent this from happening. On the contrary, it exasperates the problem.

The prior art related to penis enlargement devices employing/utilizing vacuum force (negative pressure) demonstrates several design faults. These design faults not only inhibit the effective use of such devices but also introduce the inherent risk of discomfort, pain, and potential injury even when these devices are used as intended.

Thus, there is a need for a safe, efficient, and effective vacuum chamber apparatus that can be easily secured over the penis for the purpose of applied traction without introducing several of the common design faults that result in discomfort, pain, and potential injury to the user.

### SUMMARY

While the way that the present disclosure addresses the disadvantages of the prior art will be discussed in greater detail below, in general, the present disclosure provides a vacuum chamber apparatus that can be secured over the glans of the penis for the purpose of penis enlargement.

An object of the present disclosure is to provide a vacuum chamber extension device that prevents hyper-expansion of the glans.

An object of the present disclosure is to provide a vacuum chamber extension device that evenly distributes vacuum throughout the chamber.

An object of the present disclosure is to provide a vacuum chamber extension device that eliminates obstructions that can block evacuation of the vacuum chamber.

An object of the present disclosure is to provide a vacuum chamber extension device that maximizes the compression grip on the penis shaft directly behind the glans.

An object of the present disclosure is to provide a vacuum chamber extension device that prevents the frenulum from being sucked back up into the chamber.

An object of the present disclosure is to provide a vacuum chamber extension device that minimizes cavitation within the chamber under applied traction.

The prerequisite of a properly functioning apparatus begins with a precisely fitted vacuum chamber. This is necessary to contain and protect the glans from hyper-expansion under vacuum. An accurately fitted chamber also provides the necessary friction needed for a secure grip under applied traction. Effective vacuum distribution throughout the chamber is achieved by means of a protective glans cap patterned with a network of evacuation channels. These pluralities of vacuum channels intersect with the multi-port evacuation disc which forms the interior ceiling of the vacuum chamber. This ensures free air flow during the evacuation process which cannot be interrupted or blocked under any circumstances.

The base of the vacuum chamber must provide both an air-tight seal and a firm compression grip on the penis shaft behind the glans. This prevents any air from leaking into the vacuum chamber, thereby ensuring stable vacuum levels during prolonged periods of applied traction. This also keeps the sensitive skin of the frenulum securely in place and prevents it from being sucked back up into the chamber.

The user is also instructed on the application of a novel method of locking the glans into the vacuum chamber. This method of “glans-lock” creates a powerful bond between the glans and chamber that also doesn't allow the glans to separate and form an air pocket under applied traction.

The foundation of the present invention consists of a wide size range of vacuum chambers. This sequence or series of closely configured chamber sizes can contain practically all sizes and shapes of the human penis. Each vacuum chamber is specifically sized to not only limit the expansion of the glans but also provide the necessary contact required for a friction grip on the glans and upper portion of the penis shaft. The vacuum chambers are equally proportioned with respect to the overall length and width for each size.

Once the components are fully assembled according to the user's preference for one or the other of the described embodiments, the principle of glans-lock is applied to create a solid bond between the glans, glans cap, and chamber wall. Effective glans-lock requires both external expansion of the glans that occur during evacuation of the chamber along with the internal pressure of engorgement that is achieved by manually jelqing the semi-erect penis. Without the internal pressure of engorgement, the exclusively vacuum expanded glans remains soft and spongy. In which case, as traction is applied, the glans will collapse and compress back in on itself to cavitate and form an air-pocket. The higher negative pressure generated within the air pocket causes the glans and urinary meatus to hyper-expand with a serious risk of blister formation. For additional internal pressure of engorgement, the user can initiate the arousal response to increase glans hardness, thereby ensuring maximum benefits from the glans-lock principal.

The present disclosure overcomes the existing drawbacks of the existing devices by providing a safe, easy-to-use, effective, and efficient vacuum chamber apparatus that can be secured over the glans of the penis for the purpose of penis enlargement.

### BRIEF DESCRIPTION OF THE DRAWINGS

A more complete understanding of the present disclosure may be derived by referring to the detailed description and claims when considered in connection with the figures, wherein like reference numerals refer to similar elements throughout the figures, and

FIG. 1 illustrates a cross-sectional front view of an assembled vacuum chamber extension device according to an embodiment of the present invention;

FIG. 2 illustrates an exploded view of the vacuum chamber extension device of FIG. 1, in accordance with an embodiment of the present disclosure;

FIGS. 3-13 illustrates various stages of assembly of the vacuum chamber extension device of FIG. 1;

FIG. 3 illustrates a front view of a vacuum chamber extension device in accordance with an embodiment of the present disclosure, wherein an elastic polymer sleeve is positioned on the penis shaft as depicted in the first stage of assembly of the vacuum chamber extension device;

FIG. 4 illustrates a front view of the vacuum chamber extension device of FIG. 3, wherein the glans cap is placed over the glans as depicted in the second stage of assembly of the vacuum chamber extension device; with a portion of the glans cap layered over the upper section of the elastic polymer sleeve of the vacuum chamber extension device;

FIG. 5 illustrates a front view of the vacuum chamber extension device of FIG. 4, wherein a vac-seal is now positioned over top of a portion of the glans cap as depicted in the third stage of assembly of the vacuum chamber extension device;

FIG. 6 illustrates a cross-sectional front view of the vacuum chamber extension device of FIG. 5, wherein an outer lip of the vac-seal is now folded to receive a vacuum chamber as depicted in the fourth stage of assembly of the vacuum chamber extension device;

FIG. 7 illustrates a cross-sectional front view of the vacuum chamber extension device of FIG. 6, wherein the outer lip of the vac-seal is now unfolded back to create an air-tight seal with the vacuum chamber, as depicted in the fifth stage of assembly of the vacuum chamber extension device, according to an embodiment of the present invention;

FIG. 8 illustrates a front view of the vacuum chamber extension device of FIG. 6, wherein the outer lip of the vac-seal is now unfolded to create an air-tight seal with the vacuum chamber, as depicted in the fifth stage of assembly of the vacuum chamber extension device, according to another embodiment of the present invention;

FIG. 9 illustrates various perspective views of a glans cap of the vacuum chamber extension device of FIG. 8; wherein a multi-port evacuation disc is shown as suspendably positioned on the top of the glans cap for ease of understanding;

FIG. 10 illustrates a cross-sectional perspective view of a vacuum chamber of the vacuum chamber extension device of FIG. 8; as depicted in the sixth stage of assembly of the vacuum chamber extension device;

FIG. 11 illustrates a perspective view of a vacuum chamber of the vacuum chamber extension device of FIG. 10; wherein the screw cap is shown as screwably removed from the vacuum chamber as depicted in sixth stage of assembly of the vacuum chamber extension device;

FIG. 12 illustrates a cross-sectional front view of a vacuum chamber of the vacuum chamber extension device of FIG. 11; wherein the vacuum line adapter is now inserted to fill the negative space of the adapter receptacle as depicted in the sixth stage of assembly of the vacuum chamber extension device;

FIG. 13 illustrates a cross-sectional front view of a vacuum chamber of the vacuum chamber extension device of FIG. 12; wherein the screw cap is now screwably attached to the vacuum chamber as depicted in the seventh stage of assembly of the vacuum chamber extension device;

FIG. 14 illustrates a cross-sectional front view of a vacuum chamber extension device, according to another embodiment of the present invention; and

FIG. 15 illustrates a cross-sectional front view of a vacuum chamber extension device, according to yet another embodiment of the present invention.

### DETAILED DESCRIPTION

The following description is of exemplary embodiments of the invention only, and is not intended to limit the scope, applicability, or configuration of the invention. Rather, the following description is intended to provide a convenient illustration for implementing various embodiments of the invention. As will become apparent, various changes may be made in the function and arrangement of the elements described in these embodiments without departing from the scope of the invention as set forth herein. It should be appreciated that the description herein may be adapted to be employed with alternatively configured devices having different shapes, components, attachment mechanisms and the like and still fall within the scope of the present invention. Thus, the detailed description herein is presented for purposes of illustration only and not for limitation.

Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

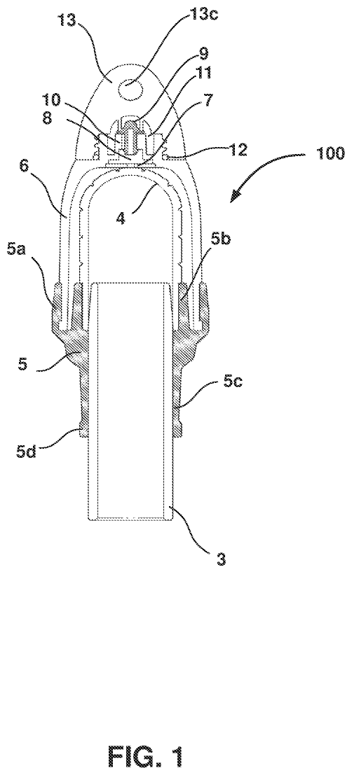

FIG. 1 illustrates a cross-sectional front view of a vacuum chamber extension device 100 in an assembled stage according to an embodiment of the present invention. As seen in FIG. 1, the vacuum chamber extension device 100 comprises an elastic polymer sleeve 3, a glans cap 4, and a vac-seal 5. The vac-seal comprises an outer lip 5a, an inner lip 5b, a thinner lower base section 5c, and an annular rim 5d. The vacuum chamber extension device 100 further comprises a vacuum chamber 6 which further comprises a multi-port disc 7, a vacuum reservoir 8, a valve 9, a valve housing 10, a receptacle 11 and a screw cap 13 comprising a center cap hole 13c for attachment of the carabiner (not shown) holding weights, the entirety of which will be described in greater detail in below description.

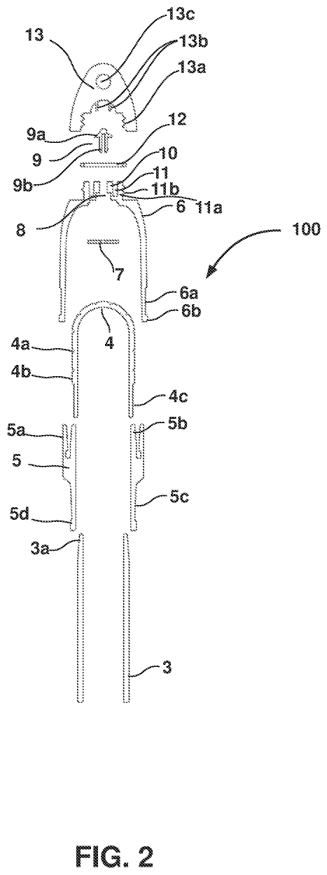

FIG. 2 illustrates an exploded view of the vacuum chamber extension device 100 of FIG. 1, in accordance with an embodiment of the present disclosure. FIG. 2 depicts an exploded view of the vacuum chamber extension device 100 according to a preferred embodiment showing the polymer sleeve 3 comprising an tapered upper section 3a, a glans cap 4 comprising:—plurality of vacuum channels 4a, a vacuum channel termination band 4b and a thinner diameter base section 4c, a vac-seal 5 which comprises:—an outer lip 5a, an inner lip 5b, a thinner lower base section 5c and an annular lip 5d, a vacuum chamber 6 which comprises:—a contoured base section 6a and an annular rim 6b, a multi-port disc 7, a vacuum reservoir 8, a valve 9 comprising a valve flap 9a, with beveled flange at the base 9b, a valve housing 10, a receptacle 11 comprising an outer wall 11a with threads 11b, an O-ring 12, a screw cap 13 comprising screw cap threads 13a, a circular valve lock post 13b and a center cap hole 13c for attachment of the carabiner (not shown), the entirety of which will be described in greater detail in below description.

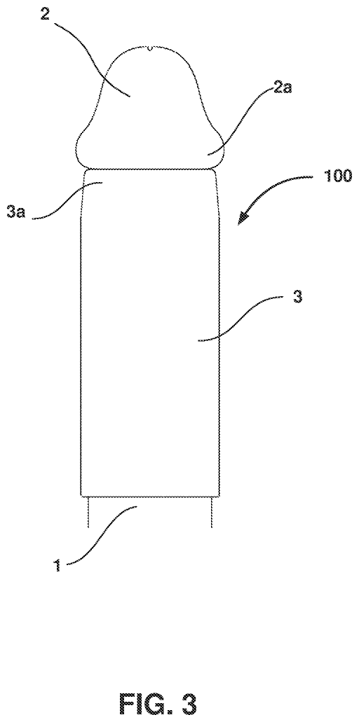

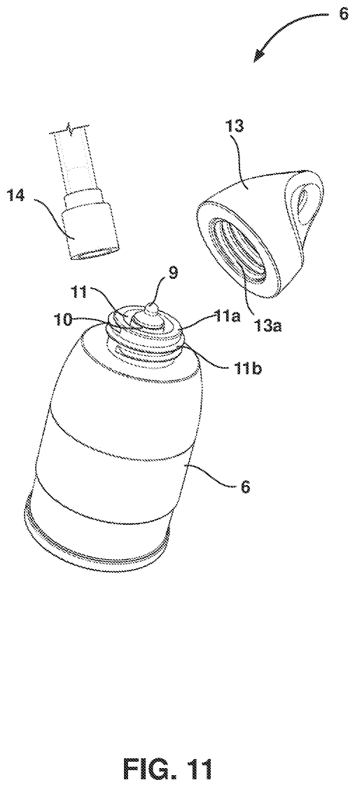

An exemplary method of assembling and/or using the vacuum chamber extension device 100 on the penis of the user will now be described with reference to FIGS. 3-15 wherein the penis of the user comprises a penis shaft 1 and a glans 2.

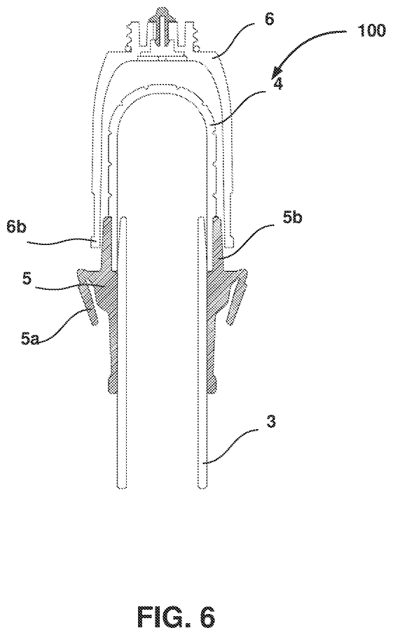

Referring to FIG. 3, Initially, in the first stage of assembly, an elastic polymer sleeve 3 is positioned on the penis shaft 1 of the user, directly behind the coronal ridge 2a of the glans 2. The elastic polymer sleeve 3 is dimensioned to have similar shape and size as the user's penis shaft 1. The elastic polymer sleeve 3 slightly compresses the penis shaft 1 to support the penis in preparation for the additional components that make up the fully assembled vacuum chamber extension device 100. The elastic polymer sleeve 3 includes a tapered upper section 3a that is configured for a compression fit as the vacuum chamber 6 (FIG. 6) is pressed down into position (not shown). However, the straight end side of the elastic polymer sleeve 3 (opposite end) can be used if a tighter fit is preferred by positioning the tapered upper section 3a down towards the base of the penis shaft 1.

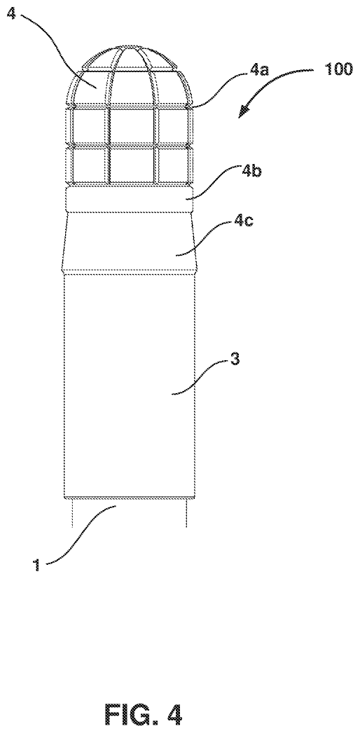

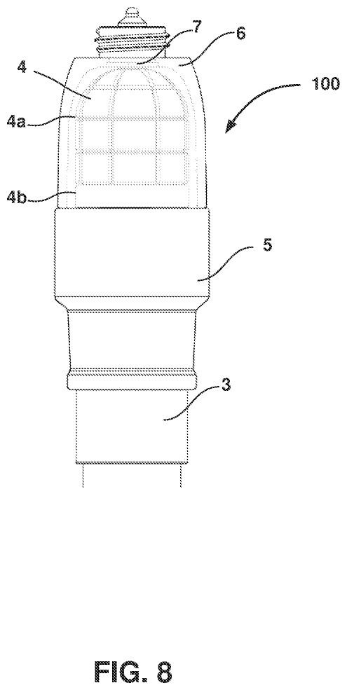

Afterwards, in the second stage of assembly, the glans cap 4 is placed over the glans 2 as shown in FIG. 4, of the vacuum chamber extension device 100, and thus, the glans 2 of the penis is protected from direct vacuum exposure by means of the glans cap 4. The thinner diameter base section 4c of the glans cap 4 is placed directly behind the coronal ridge 2a of the glans 2 (FIG. 3) and thus partially layered over top of the elastic polymer sleeve 3 already in position on the penis shaft 1. This combination of components helps to prepare for an air-tight seal when the vacuum chamber 6 (FIG. 8) is secured into position. This arrangement effectively secures the frenulum in place as well thereby preventing the frenulum from being sucked up into the vacuum chamber 6 (FIG. 8) under vacuum with applied traction.

In another embodiment, the assembly of the vacuum chamber extension device 100 can also be reversed. The assembly of the vacuum chamber extension device 100 could begin with the glans cap 4 first placed over the glans 2, then followed by placing the elastic polymer sleeve 3 on the penis shaft 1. The polymer sleeve 3 is layered over top (not shown) of the thinner diameter base section 4c of the glans cap 4.

The plurality of vacuum channels 4a are configured for evacuation through the upper portion of the vacuum chamber 6 (FIG. 8) during the evacuation process. The vacuum channel termination band 4b functions to isolate the vacuum within the plurality of vacuum channels 4a surrounding the glans 2 while ensuring that no air can enter or exit the vacuum chamber 6.

The precision fitted glans cap 4 is dimensioned to compress the glans 2 yet allows for the glans 2 to expand under vacuum to contact the interior of the wall of the vacuum chamber 6 (FIG. 8). Contact with the interior wall (not shown) of the vacuum chamber 6 contains the glans 2 to prevent hyper expansion while providing the necessary friction needed to secure the glans 2 within the vacuum chamber 6 under applied traction.

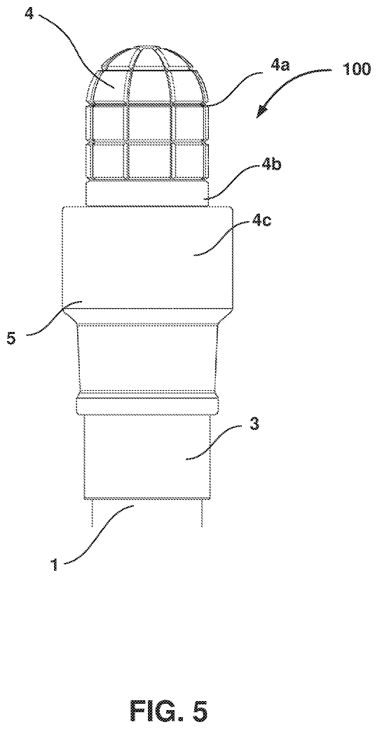

Afterwards, in the third stage of assembly as seen in FIG. 5, the vac-seal 5 is positioned over top of the thinner diameter base section 4c (FIG. 4) of the glans cap 4 in alignment with the proximal edge of the vacuum channel termination band 4b and upper distal section of the elastic polymer sleeve 3 layered on top of the penis shaft 1.

Afterwards, in the fourth stage of assembly as seen in FIG. 6, the outer lip 5a of the vac-seal 5 is folded back to allow receiving of the annular rim 6b of the vacuum chamber 6. The inner lip 5b is wedge shaped to provide a progressively tighter compression grip on the penis shaft 1 as the hard plastic of the annular rim 6b of the vacuum chamber 6 is cinched down into the space that is formed between the outer lip 5a and the inner lip 5b of the vac-seal 5. This helps to further stabilize the frenulum against the penis shaft 1 thereby preventing the frenulum from being inadvertently sucked back up inside the glans cap 4 under vacuum with applied traction.

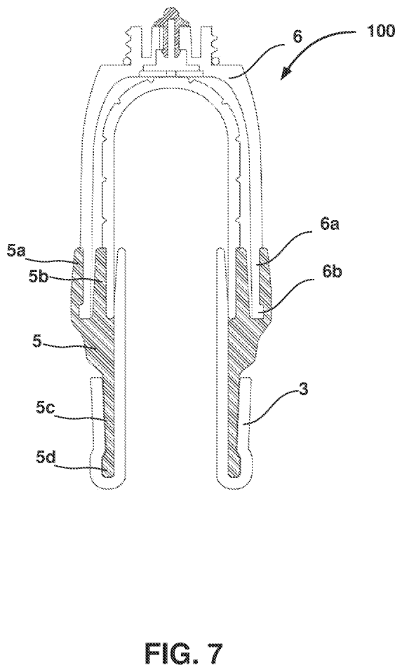

Afterwards, in the fifth stage of assembly as seen in FIG. 7, to create an air-tight seal, the outer lip 5a is unfolded, upright back into place, thereby air-tight sealing the vacuum chamber 6 relative to the vac-seal 5. The inner surface of the outer lip 5a is contoured to match the contours of the contoured base section 6a and an annular rim 6b of the vacuum chamber 6. The underlying length of elastic polymer sleeve 3 is folded back over the annular rim 5d as seen in FIG. 7 to cover the thinner lower base section 5c. This folding of the elastic polymer sleeve 3 creates a triple layer that effectively increases the compression grip on the upper penis shaft 1. As shown in FIG. 7, the vacuum chamber 6 at least partially encloses (surrounds) the glans cap 4. Further, there is a sufficient gap between the vacuum chamber 6 and the glans cap 4 as shown in FIG. 7 to facilitate easy insertion of the vacuum chamber 6 before evacuation.

In another embodiment, the underlying length of elastic polymer sleeve 3 can be left covering the penis shaft 1 as seen in FIG. 6 and FIG. 8.

Referring to FIG. 7 and FIG. 8, the glans 2 (FIG. 3) is contained within the glans cap 4. During evacuation, both the glans 2 and glans cap 4 expand to completely fill the interior of the vacuum chamber 6 (not shown in figures). A plurality of vacuum channels 4a allows for unhindered evacuation throughout the vacant space within the vacuum chamber 6. Under vacuum, the glans cap 4, expands to fill the vacuum chamber 6, thus making contact with the multi-port evacuation disc 7, which acts as the ceiling at the interior distal end of the vacuum chamber 6. The plurality of vacuum channels 4a are effectively sealed off by the vacuum channel termination band 4b to prevent air from entering or exiting the vacuum chamber 6.

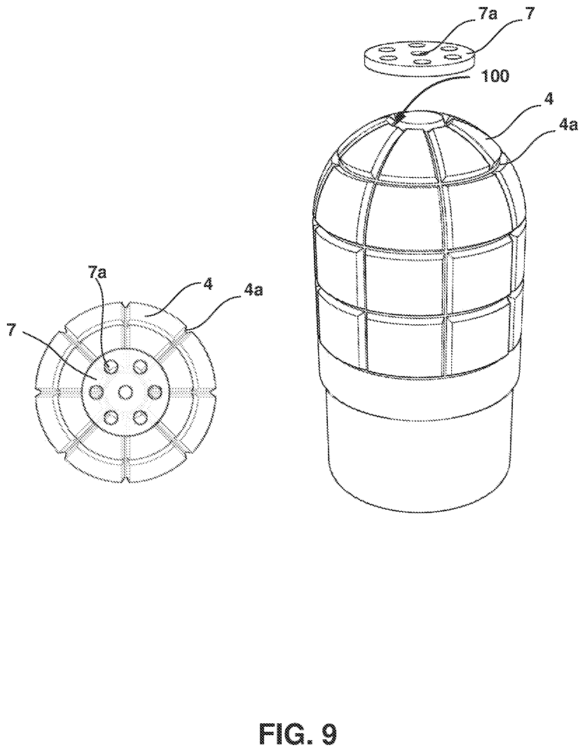

FIG. 9 illustrates various perspective views of a glans cap 4 of the vacuum chamber extension device 100 of FIG. 8; wherein a multi-port evacuation disc 7 is shown as suspendably positioned above the glans cap 4 for ease of understanding.

Referring to FIG. 9 the glans cap 4 comprises a plurality of vacuum channels 4a, intersecting with the multi-port disc 7, through a plurality of evacuation ports 7a. As seen in left hand side FIG. 9 (top view), the pattern of plurality of vacuum channels 4a of the glans cap 4 and the plurality of evacuation ports 7a of the multi-port evacuation disc 7 are arranged offset to effectively maintain the unrestricted air flow needed to reach the vacuum levels required within the vacuum chamber 6 (FIG. 8). In other words, some of the vacuum channels 4a of the glans cap 4 and some of the evacuation ports 7a of the multi-port evacuation disc 7 are not arranged in a straight line when seen from the top view in the left hand side FIG. 9. In contrast, the referenced examples of prior art utilize single port designs that are invariably blocked as the glans 2 expands during the evacuation process. Once blocked, any increase in negative pressure is isolated outside the chamber to produce a false reading on the vacuum gauge (not shown).

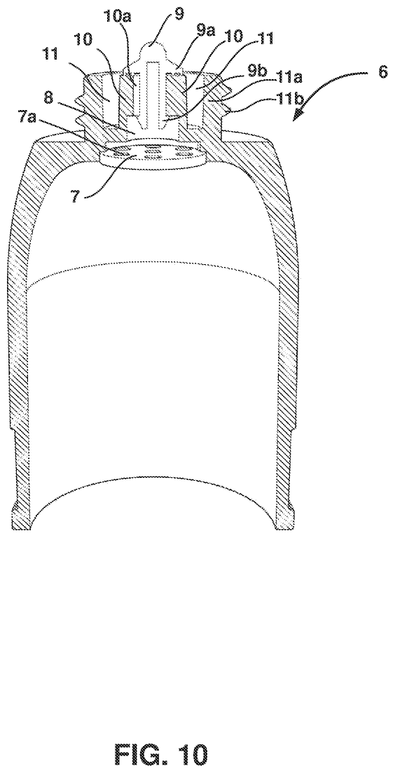

FIGS. 10-12 illustrates a cross-sectional perspective view, a perspective view and a cross-sectional front view of a vacuum chamber 6 of the vacuum chamber extension device 100 of FIG. 8 respectively as depicted in the sixth stage of assembly of the vacuum chamber extension device 100. Referring to FIGS. 10-12, during evacuation, air passes through the multi-port disc 7 into the vacuum reservoir 8. The wider diameter of the vacuum reservoir 8 allows for a bigger multi-port disc 7 comprising a plurality of evacuation ports 7a and also provides the necessary clearance required to ensure unhindered valve function. A valve 9 is secured in position in the valve housing 10 above the vacuum reservoir 8. The interior of the valve housing 10 includes a plurality of vertical channels 10a that are slightly wider than the circumference of the beveled flange of a valve base 9b of the valve 9 to allow free unrestricted passage of air. During evacuation, the valve flap 9a lifts to allow air to pass with each suction action of the vacuum pump (not shown). The valve flap 9a then returns flush against the flat distal surface of the valve housing 10 to establish a positive seal, particularly after negative pressure has been introduced within the vacuum reservoir 8. The exterior wall of the valve housing 10 serves as the inner wall of a receptacle 11. The receptacle further comprises an outer wall 11a. Thus, the receptacle 11 so formed is a circular hollow space defined by the interior of an outer wall 11a and the exterior wall of the valve housing 10. The receptacle 11 so formed is configured to receive a vacuum line adapter 14 that is fluidly connected to a vacuum pump (not shown in figures). The shape of the receptacle 11 is configured to form an airtight seal upon insertion of the vacuum line adapter 14 after the vacuum line adapter 14 is placed over top of the valve 9 and valve housing 10 to form a compression fit inside the receptacle 11. The exterior of the outer wall 11a of the receptacle 11 includes threads 11b to removably (screwably) engage with the corresponding threads 13a of the screw cap 13 for secure attachment onto the vacuum chamber 6.

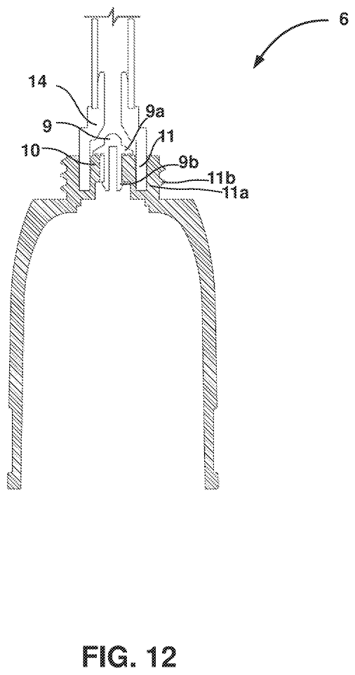

Referring to FIG. 12, the vacuum line adapter 14 is inserted to fill the negative space of the receptacle 11 with compression fit for an air-tight seal. The valve 9 is secured within the valve housing 10, wherein the valve housing 10 is of a slightly wider diameter than the outer circumference of the valve flap 9a. This ensures that the valve flap 9a is not inadvertently lifted upon removal of the vacuum line adapter 14 to break the seal with subsequent loss of vacuum.

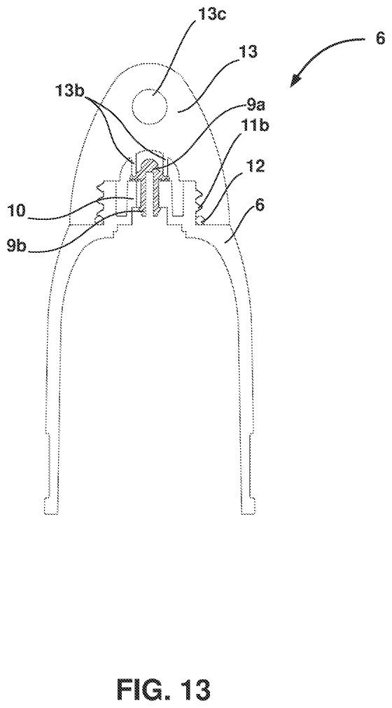

Afterwards, in the seventh stage of assembly as seen in FIG. 13, the screw cap 13 is now screwably attached to the vacuum chamber 6. The screw cap 13 includes an interior circular wall that acts as a valve lock post 13b to compress the valve flap 9a down firmly against the flat distal surface of the valve housing 10. This is necessary to ensure an airtight seal for the duration of use, which may last 5-6 hours or more when “all day stretching”. It should be understood that a variety of valves 9 with different mechanisms can also serve to maintain the negative pressure of vacuum within the vacuum chamber 6. An O-ring 12 is positioned at the base of the threads 11b of the outer wall 11a of the receptacle 11. The O-ring 12 acts as a cushion for the screw cap 13 when the screw cap 13 is cinched down in place on the flat distal surface of the vacuum chamber 6. The upper section of the screw cap 13 includes a center cap hole 13c to receive a carabiner with a suspension strap (not shown) to hang weight for applied traction.

Further, it should be obvious to the one skilled in the art that the center cap hole 13c could be used to attach any other quick-connect coupling such as pins, metal loop, suspension strap, band, bayonet locks, hooks, chains and so on.

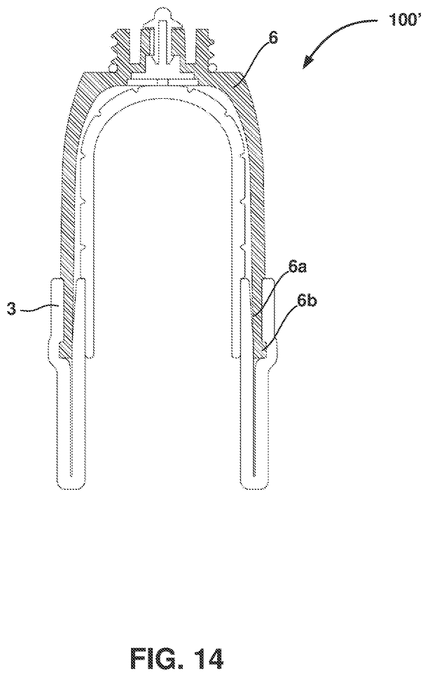

FIG. 14 illustrates a cross-sectional front view of a vacuum chamber extension device 100′, according to another embodiment of the present invention. The vacuum chamber extension device 100′ utilizes/employs a substantially smaller, more discreet vacuum chamber 6 that does not require the vac-seal to be included in the assembly. After insertion of the glans 2 (not shown), within glans cap 4, into the vacuum chamber 6, the length of elastic polymer sleeve 3 is folded back up and over (see arrows in FIG. 14) the exterior of the contoured base section 6a and an annular rim 6b to create an air-tight seal. This embodiment is particularly useful when applying traction of low-intensity/long-duration for “all day stretching” (ADS). It should be fairly understood that other components of the vacuum chamber extension device 100′ are quite similar to components of the vacuum chamber extension device 100 (FIG. 1-13) with little to no variation.

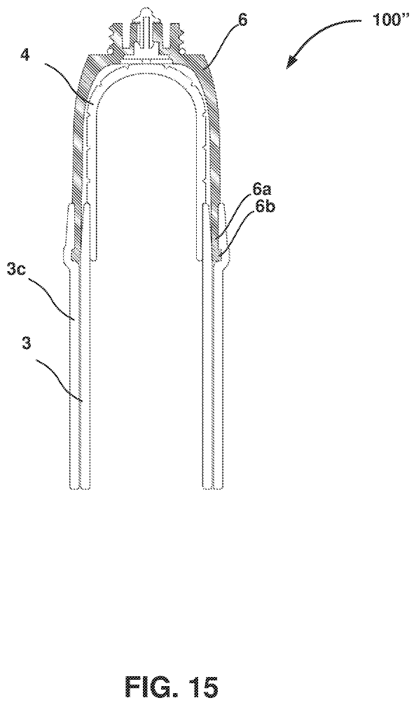

FIG. 15 illustrates a cross-sectional front view of a vacuum chamber extension device 100″, according to another embodiment of the present invention. The vacuum chamber extension device 100′ utilizes/employs a vacuum chamber 6 and two elastic polymer sleeves 3, 3c such that the vacuum chamber extension device 100′ does not require the vac-seal to be included in the assembly. After insertion of the glans 2 (not shown) within glans cap 4 into the vacuum chamber 6, the length of elastic polymer sleeve 3 is left covering the length of the penis shaft 1. For added security, a second elastic polymer sleeve 3c is layered over top of the first polymer sleeve 3. This provides for a more flexible airtight seal around the contoured base section 6a and an annular rim 6b of the vacuum chamber 6. This is particularly useful when traction is applied horizontally around the waist by means of an elastic belt (not shown) to maintain a secure air-tight seal. In that case, bending of the penis around the hip risks compromising the seal created by the single elastic polymer sleeve 3 folded over the annular rim 6b and contoured base section 6a of the chamber (FIG. 14) with subsequent loss of vacuum. It should be fairly understood that other components of the vacuum chamber extension device 100″ are quite similar to components of the vacuum chamber extension device 100 (FIG. 1-13) with little to no variation.

Finally, while the present invention has been described above with reference to various exemplary embodiments, many changes, combinations, and modifications may be made to the exemplary embodiments without departing from the scope of the present invention. For example, the various components may be implemented in alternative ways. These alternatives can be suitably selected depending upon the particular application or in consideration of any number of factors associated with the operation of the device. In addition, the techniques described herein may be extended or modified for use with other types of devices. These and other changes or modifications are intended to be included within the scope of the present invention.

What is claimed is:

1. A vacuum chamber extension device (100) configured for penis enlargement of a user, the vacuum chamber extension device (100), comprising:
an elastic polymer sleeve (3) configured to be positioned on the penis shaft (1);
a glans cap (4) configured to be placed over the glans (2), wherein the glans cap (4) comprises a plurality of vacuum channels (4a), and a thinner diameter base section (4c) that is configured to cover the upper portion of the penis shaft (1);
a vacuum chamber (6) configured to at least partially enclose the glans cap (4), wherein the vacuum chamber (6) comprises a multi-port disc (7), a vacuum reservoir (8), a valve (9), a valve housing (10) and a receptacle (11) comprising threads (11b) on the exterior of an outer wall (11a) of the receptacle (11);
Wherein the exterior wall of the valve housing (10) and the receptacle (11) are configured to receive a vacuum line adapter (14);
Wherein the plurality of vacuum channels (4a) of the glans cap (4) are arranged offset relative to a plurality of evacuation ports (7a) of the multi-port evacuation disc (7); and
a screw cap (13) comprising threads (13a) that are configured to removably engage with the threads (11b) of the outer wall (11a) of the receptacle (11).

2. The vacuum chamber extension device (100) according to claim 1, wherein the screw cap (13) comprises a center cap hole (13c) for attachment of a carabiner.

3. The vacuum chamber extension device (100) according to claim 1, wherein the screw cap (13) includes an interior circular wall that acts as a valve lock post (13b) to compress a valve flap (9a) of the valve (9) down firmly against the flat distal surface of the valve housing (10).

4. The vacuum chamber extension device (100) according to claim 1, wherein a thinner diameter base section (4c) of the glans cap (4) is partially layered over top of the elastic polymer sleeve (3).

5. The vacuum chamber extension device (100) according to claim 1, wherein the polymer sleeve (3) is partially layered over a thinner diameter base section (4c) of the glans cap (4).

6. The vacuum chamber extension device (100) according to claim 1, wherein a vacuum channel termination band (4b) of the glans cap (4) is configured to isolate the vacuum within the plurality of vacuum channels (4a).

7. The vacuum chamber extension device (100) according to claim 1, wherein a vac-seal (5) is positioned over top of a thinner diameter base section (4c) of the glans cap (4) and tapered upper section (3a) of the elastic polymer sleeve (3), wherein the vac-seal (5) comprises an outer lip (5a), an inner lip (5b), a thinner lower base section (5c) and an annular lip (5d).

8. The vacuum chamber extension device (100) according to claim 1, wherein both the glans (2) and glans cap (4) are configured to expand to completely fill the interior of the vacuum chamber (6) during evacuation, such that the glans cap (4) makes contact with the multi-port evacuation disc (7) and interior walls of the vacuum chamber (6) during evacuation.

9. The vacuum chamber extension device (100) according to claim 1, wherein the interior of the valve housing (10) includes a plurality of vertical channels (10a) that are dimensioned to be slightly wider than the circumference of a beveled flange of a valve base (9b).

10. The vacuum chamber extension device (100) according to claim 1, wherein the vacuum line adapter (14) is fluidly connected to a vacuum pump.

11. The vacuum chamber extension device (100) according to claim 1, wherein a valve flap (9a) of the valve (9) is configured to be lifted to allow suction of air through valve (9).

12. The vacuum chamber extension device (100) according to claim 1, wherein a second elastic polymer sleeve (3c) is configured to be layered over top of the elastic polymer sleeve (3).

13. A method for assembling and using a vacuum chamber extension device (100) configured for penis enlargement of a user, the method comprising the steps of:
positioning a polymer sleeve (3) on the penis shaft (1) and positioning the glans cap (4) over the glans (2), wherein the glans cap (4) comprises vacuum channel termination band (4b), a thinner diameter base section (4c);
positioning a vac-seal (5) over top of the thinner diameter base section (4c) of the glans cap (4) and tapered upper section (3a) of the elastic polymer sleeve (3); wherein the vac-seal (5) comprises an outer lip (5a);
folding the outer lip (5a) of the vac-seal (5) to allow receiving of an annular rim (6b) of a vacuum chamber (6), wherein the vacuum chamber (6) comprises a multi-port disc (7), a vacuum reservoir (8), a valve (9), a valve housing (10) and a receptacle (11) comprising threads (11b) on the exterior of an outer wall (11a) of the receptacle (11);
unfolding the outer lip (5a) of the vac-seal (5), thereby enabling air-tight sealing of the vacuum chamber (6) relative to the vac-seal (5);
evacuating the vacuum chamber (6) by fluidly connecting a vacuum line adapter (14) to the receptacle (11), thereby enabling expansion of the glans cap (4) such that the glans cap (4) makes contact with the multi-port evacuation disc (7) during evacuation; and
removably engaging threads (13a) of a screw cap (13) with the threads (11b) of the outer wall (11a) of the receptacle (11).

14. The method for assembling and using a vacuum chamber extension device (100) according to claim 13, further comprises an additional step of:—attaching a carabiner to a center cap hole (13c) of the screw cap (13).

15. The method for assembling and using a vacuum chamber extension device (100) according to claim 13, wherein the vacuum line adapter (14) is fluidly connected to a vacuum pump.

16. The method for assembling and using a vacuum chamber extension device (100) according to claim 13, wherein a vacuum channel termination band (4b) of the glans cap (4) is configured to isolate the vacuum within the plurality of vacuum channels (4a).

17. The method for assembling and using a vacuum chamber extension device (100) according to claim 13, further comprises an additional step of:—positioning the glans cap (4) relative to the elastic polymer sleeve (3) such that a thinner diameter base section (4c) of the glans cap (4) is partially layered over top of the elastic polymer sleeve (3).

18. The method for assembling and using a vacuum chamber extension device (100) according to claim 13, further comprises an additional step of:—positioning the elastic polymer sleeve (3) relative to the glans cap (4) such that the polymer sleeve (3) is partially layered over a thinner diameter base section (4c) of the glans cap (4).

19. The method for assembling and using a vacuum chamber extension device (100) according to claim 13, wherein the interior of the valve housing (10) includes a plurality of vertical channels (10a) that are dimensioned to be slightly wider than the circumference of a beveled flange of a valve base (9b).
